(19) United States
(12) Patent Application Publication
(10) Pub. No.: US 2005/0131302 A1
(43) Pub. Date: Jun. 16, 2005

(54) Ultrasonic probe having a selector switch

(76) Inventor: McKee D. Poland, Andover, MA (US)

Correspondence Address: PHILIPS INTELLECTUAL PROPERTY & STANDARDS, P.O. BOX 3001, BRIARCLIFF MANOR, NY, 10510, US

(21) Appl. No.: 11/009,316

(22) Filed: Dec. 10, 2004

Related U.S. Application Data
(60) Provisional application No. 60/529,787, filed on Dec. 16, 2003.
(60) Provisional application No. 60/615,426, filed on Oct. 1, 2004.

(51) Int. Cl. A61B 8/14

(52) U.S. Cl. 600/459

(57) ABSTRACT

An ultrasonic probe having a selector switch and a housing is provided. The ultrasonic probe further includes an ultrasonic transducer assembly and associated circuitry. A beamformer may be included in the ultrasonic probe. The selector switch has at least two user-selectable positions or states. The selector switch and the associated circuitry control an output acoustic beam of the ultrasonic imaging apparatus in accordance with the user-selectable position or state.

### CROSS REFERENCE TO RELATED CASES

Applicant claims the benefit of Provisional Application Ser. No. 60/529,787, filed 16 Dec. 2003, and Provisional Application Ser. No. 60/615,426, filed 1 Oct. 2004.

### BACKGROUND OF THE INVENTION

1. Field of the Invention

The present invention relates generally to ultrasonic imaging systems. More particularly, it relates to ultrasonic imaging systems with an ultrasonic probe having a selector switch for controlling characteristics of the acquired ultrasonic image.

2. Description of the Related Art

Ultrasonic transducer probes transmit and receive ultrasound energy in any diagnostic ultrasound medical imaging system. Ultrasound medical imaging systems are used in many medical applications and, in particular, for the non-invasive acquisition of images of organs and conditions within a patient, e.g., fetuses, the heart. Ultrasonic transducer probes are generally hand held, but vary significantly in accordance with their intended imaging application. There are transthoracic transducer probes, transesophageal echocardiographic (TEE) transducer probes, vascular transducer probes, intra-cardiac transducer probes, etc.

Ultrasonic transducer probes are formed with one-dimensional and two-dimensional transducer arrays including a plurality of acoustic elements arranged in a linear or planar configuration. The acoustic elements are typically piezo electric. They mechanically deform in response to electrical drive signals, creating tiny acoustic waves which are coupled from the transducer probe into the medium, which is typically a human body. The acoustic waves propagate away from the transducer, creating echoes at the interfaces between structures in the medium that have differing acoustic index. The receive echoes propagate back through the medium and impinge upon the elements of the transducer array, deforming the array elements and creating tiny electrical receive signals. By adjusting the time delays of the electrical drive signals and of the electrical receive signals on elements of a one-dimensional or two-dimensional transducer array, beam steering and focusing of transmitted and received ultrasound energy is achieved. The aforementioned time delay adjustments control both the propagation of the transmitted ultrasonic energy and the path of maximum sensitivity to received echo signals, such that the beams formed thereby are steered along a chosen locus of sample points. The locus of points is referred to as a scan line.

For each scan line, there is a transmit phase and a corresponding receive phase. In the transmit phase, each element from a chosen set of elements forming the transmit aperture is driven electrically to produce an acoustic transmit pulse. The transmit drive signals are time delay adjusted with respect to each other by a scan controller so as to create a particular path of maximum acoustic power propagation in the medium. The resulting three-dimensional profile of transmitted acoustic power in the medium is referred to in the art as the transmit beam, and represents a physical summation of the acoustic contributions of the elements chosen for transmission. Likewise, for the receive phase of the scan line, a receive beam is formed by adjusting the time delays of the received electrical echo signals from a chosen set of elements of the acoustic array, the chosen set forming the receive aperture, and summing the contributions from each of the chosen receive elements. Whereas the summation of the transmit signals from elements happens in the medium according to physical laws and the structure of the medium in response to the transmit pulses, the summation of the receive signals from received echoes is performed by the ultrasound system. The time adjustment of individual received signals from elements before summation determines the locus of points along the receive path of the scan line from which the most acoustic energy is collected in summation. The three-dimensional profile of the received acoustic power in the medium along the scan line is referred to as the receive beam, and represents the contributions of the received, delayed and summed signals of the elements chosen to serve in the receive phase of the scan line.

The process of adjusting the time delays and forming the sums of signals to or from the array of elements is referred to as beamforming. Transmit beamforming applies the transmit phase of the scan line, wherein the delay adjustments are applied to element drive signals. Receive beamforming applies to the receive phase of the scan line, wherein the delay adjustments are applied to the electrical signals produced by elements as receive echoes impinge upon the transducer. By altering the time delays of the received element signals at various points in time during the receive phase, the focus and steering of the summed receive beam is updated dynamically, allowing the scan line's receive focus to follow the incoming echo path and to vary the steering angles of the scan line during the course of reception. The aforementioned time delay alterations are referred to as dynamic receive beamforming.

It is possible to form multiple receive signal summations, using different sets of dynamically altered receive delays, thus forming multiple receive beams simultaneously in a given receive phase of a scan line. This technique is sometimes referred to as receive parallelism, and provides a means of interrogating more of the medium per scan line than by using just a single receive beam. A set of scan lines is processed by the ultrasound system into image data which is then displayed. A single set of scan lines forming an image is referred to as a scan frame, and represents one image update on the display. The system frame rate, that is, the rate at which the display is updated with new ultrasound images, depends on the duration of individual scan lines as well as how many are used in the scan frame. By employing the aforementioned technique of receive parallelism, fewer scan lines may be utilized to generate an image, thereby desirably increasing frame rate. Alternatively, for a given frame rate derived from a given number of component scan lines, parallelism allows more receive beams to be created, thus more closely spaced interrogation of the medium, and thus finer image resulotion. Typically, each transmit beam and its corresponding receive beams are chosen to be congruent or nearly so, and the receive beams are dynamically focused and steered so that they follow the path of the receive echoes in scan lines that are straight or nearly straight.

A recent technological advance in the art of beamforming is microbeamforming, sometimes referred to also as sub-array beamforming. In newer transducers, especially those that include multi-dimensional arrays, comprising hundreds or thousands of acoustic elements, the task of driving transmit pulses to large numbers of elements, and the corresponding task of dynamically beamforming the receive signals from large numbers of elements makes for prohibitively complex and expensive beamformers. Microbeamforming solves this problem by providing a means of grouping array elements into clusters, or sub-arrays, that require similar transmit and receive operation, and beamforming the groups locally, typically within the ultrasound probe itself, producing inputs and outputs from the sub-arrays that may then be treated as inputs and outputs of larger virtual elements by a conventional transmit/receive beamformer. Microbeamforming thus greatly reduces the cost and complexity of the ultrasound system, and makes practical the usage of transducer arrays containing thousands of acoustic elements. Microbeamforming may be performed in successive stages in an ultrasound system, each stage grouping the inputs and outputs of the previous stage, thus exponentially reducing the effective number of system elements handled at the outermost level of the beamformer. Microbeamforming may be employed in conjunction with receive beam parallelism.

Transducer probes which employ one-dimensional arrays of acoustic elements are generally limited to steering scan lines in a single plane. The focus and beam shape of the transmit and receive beams out-of-plane is typically controlled by a fixed mechanical lens. Though frequently referred to as one-dimensional, so-called “curved linear array” (CLA) probes, strictly speaking, arrange elements in two dimensions along a curved line. Nevertheless, such probes share the same limitations as flat one-dimensional arrays: they may only steer beams in a single plane. One-dimensional array probes may be mounted on a mechanical rotating or oscillating means in order to automatically interrogate a rotating or oscillating image plane in the medium. However, the rotating/oscillating means adds complexity, fragility, and expense to the system, and limits the rate at which a volume in the medium can be scanned due to limited the velocity at which the mechanical movement can be actuated. Newer multi-dimensional arrays, made practical by microbeamforming as explained heretofore, contain elements arranged in 2 or 3 dimensions such that the scan lines they produce may be rapidly steered in multiple distinct planes, or in general, in any direction within a three-dimensional volume, by changing only the transmit and receive beamforming delays. Thus beam steering on multi-dimensional arrays, whether in the transmission or receipt, provides that the ultrasonic energy may be directed in any orientation within a volume whose boundaries are dictated only by the practical electro-acoustic limits of the array. That is, the ability of such a transducer probe to image a volume is directly related to the characteristics of the multi-dimensional transducer array, such as element pitch, number of elements, resonant frequency, maximum drive voltage, etc.

TEE probes include a transducer array arranged in a probe shaft adapted to be inserted into a patient's body for cardiac imaging, with a “mid-handle” connected to the probe shaft (outside the body) at one end of the mid-handle and a cable connected to the processing unit at the other end of the mid-handle. The processing unit is typically controlled by controls on a control panel, and provides images to an associated display device (e.g. a monitor). Controls are often positioned on the mid-handle to enable mechanically or electrically actuated adjustment of the articulation and rotational position of the tip of the transducer probe.

Transthoracic transducer probes typically include a one-dimensional or two-dimensional element array positioned in a handle, which is connected to a processing unit via a cable. The processing unit is controlled using controls disposed on a control panel, and provides images to a display device.

It has been a drawback of transducer probe technology that operational modes of the ultrasound imaging system were not normally found in the handle. That is, the ultrasound clinician was required to access the control panel in order to switch between imaging modes, e.g., to switch between a 2-D mode and a 3-D mode. Such control panel access is interruptive to an examination, requiring the clinician to shift his/her body, and possibly remove his/her hands from the transducer probe, often resulting in a need for repositioning of the transducer probe. U.S. Provisional application No. 60/477,632, filed Jun. 11, 2003, commonly owned and incorporated by reference herein, attempts to remedy such drawbacks by disclosing an ultrasound transducer probe with a control system incorporated into the handle to enable easy access to system controls and image-optimizing controls. For example, the transducer probe controls may allow a clinician to toggle easily between 2-D and 3-D modes.

Conventional ultrasonic imaging systems often include a positioning device such as a trackball, or other user-interface located on the system unit for controlling characteristics of the acoustic beam and therefore the acquired ultrasonic image, where the operator adjusts the acquired image by actuating the trackball on the system unit. U.S. Provisional application No. 60/477,632 teaches placing such a positioning device or trackball in the transducer probe in the case of a transthoracic probe, or in mid-handle in the case of a TEE probe. Consequently, not only can the mode of operation and therefore images obtained be controlled at the transducer probe itself, i.e., by the controls disposed therein, but also the position of indicators in the image.

While the aforementioned inventive ability to control certain ultrasound system operations directly at the transducer probe is marked improvement in ultrasound examination ergonomics, such invention, and other related ultrasound transducer probe technology, does not go far enough. That is, newer emerging transducer probe technology, such as multidimensional transducer arrays, and their controls, and microbeamforming means located within the transducer probe would be well served if controllable directly at the transducer probe itself. Accordingly, the present invention discloses apparatus and methods for controlling multidimensional transducer arrays, and their unique imaging abilities, as well as aspects enabled by microbeamforming, via controls located in the transducer probe handle, showing a marked improvement in the art. For example, the invention provides for the clinician to make adjustments easily, ergonomically, and efficiently to the imaging mode and/or scanned image position available with newly developed multidimensional transducer array technology and microbeamforming technology, using controls located at the transducer probe itself. The invention, therefore, greatly improves on the ability of the ultrasound clinician to concentrate more on the job at hand, maintain better control of the examination (e.g., minimizing re-adjustment), and more readily and expediently acquire useful and accessible image data.

### SUMMARY OF THE INVENTION

An ultrasonic transducer probe having a selector switch and controls built in for controlling imaging processes utilizing multidimensional transducer array technology and/or microbeamforming, thereby controlling the characteristics of the acquired ultrasonic image is hereinafter disclosed. In particular, the apparatus includes a housing, an ultrasonic probe having an ultrasonic transducer assembly, user controls which may include a selector switch having at least two user-selectable positions or states, and/or a positional device, and associated circuitry. The ultrasonic transducer assembly includes a plurality of acoustic elements configured and arranged in a multidimensional array, which is designed to fit within the housing of the ultrasonic probe. Each of the acoustic elements in the multidimensional transducer array is capable of generating an acoustic pulse and/or receiving an echo signal, and is controlled using microbeamforming technology. That is, a microbeamformer is coupled to the array and drives the acoustic elements included in the multidimensional array. The operation of the array is controllable via the probe handle controls, which select the imaging modes and scanning parameters of the system. The microbeamformer further includes associated circuitry capable of controlling the placement of acoustic transmit and receive beams by generating control signals in cooperation with a provided user interface, thereby controlling the acquired field of view. The associated circuitry of the ultrasonic probe may provide that the microbeamformer generates and controls the acoustic beam in accordance with at least one of the at least two user-selectable states. A signal processor is coupled to the array for processing at least one echo signal to form at least one image signal. A display operatively coupled to the signal processor is further included for displaying data corresponding to the at least one image signal. A storage device may be provided for storing and/or retrieving data corresponding to the at least one image signal. A communication device may be provided for transferring image data and associated data such as measurements, operating conditions, and image time stamps to a separate and/or remote system for storage and deferred analysis.

In one embodiment, an ultrasonic imaging apparatus including an ultrasonic probe having a selector switch and controls built in for controlling imaging processes and utilizing multidimensional transducer array technology, and microbeamforming, thereby controlling the characteristics of the acquired ultrasonic image, is hereinafter disclosed. A housing is provided. The ultrasonic imaging apparatus further includes an ultrasonic transducer assembly configured and adapted to fit within the housing, which includes user controls and a selector switch having at least two user-selectable positions or states, and/or a positional device, and associated circuitry. The ultrasonic transducer assembly includes a plurality of acoustic elements configured and arranged in a multi-dimensional array, which is designed to fit within the housing of the ultrasonic probe. Each of the acoustic elements in the multidimensional transducer array is capable of generating and/or receiving at least one echo signal. Groups of at least two acoustic elements in the multidimensional transducer array are capable of generating transmit and receive acoustic beams in a plurality of scan line directions. At least one scan line in at least one direction is generated by the transducer assembly and associated circuitry to form at least one image. The ultrasonic imaging apparatus also includes associated circuitry operatively coupled to the ultrasonic transducer assembly and the handle controls, facilitating the user's control of the at least one acoustic beam, and thereby the control of the at least one image produced, by at least one of the parameters transmit voltage, number of transmit cycles per pulse, transmit frequency, transmit focus, transmit aperture and apodization, transmit pulse waveform shape, transmit scan line direction and origin, receive aperture and apodization, receive scan line direction and origin, receive parallelism, receive filtering and echo envelope detection, Doppler ensemble processing, etc.

### BRIEF DESCRIPTION OF THE DRAWINGS

The foregoing objects and advantages of the present invention may be more readily understood by one skilled in the art with reference being had to the following detailed description of preferred embodiments thereof, taken in conjunction with the accompanying drawings in which:

FIG. 1 illustrates an ultrasonic imaging system having an ultrasonic transducer probe which includes a multi-dimensional transducer array and microbeamforming circuitry in accordance with the present invention;

FIG. 2 is a perspective view of an ultrasonic probe having microbeamforming circuitry, a transducer array, and a selector switch for use in the ultrasonic imaging system of FIG. 1;

FIG. 3 is a plan view of a transesophageal echocardiographic ultrasonic transducer probe having microbeamforming circuitry, a transducer array, and a selector switch for use in the ultrasonic imaging system of FIG. 1; and

FIG. 4 is a perspective view a multidimensional transducer array of the ultrasonic imaging system of FIG. 1, showing two sets of scan lines in planes which vary in elevation angle.

FIG. 5 is a perspective view a multidimensional transducer array of the ultrasonic imaging system of FIG. 1, showing two sets of scan lines in planes which vary in rotation angle.

### DETAILED DESCRIPTION OF THE PREFERRED EMBODIMENTS

Several embodiments of the present invention are hereby disclosed in the accompanying description in conjunction with the figures. Preferred embodiments of the present invention will now be described in detail with reference to the figures wherein like reference numerals identify similar or identical elements.

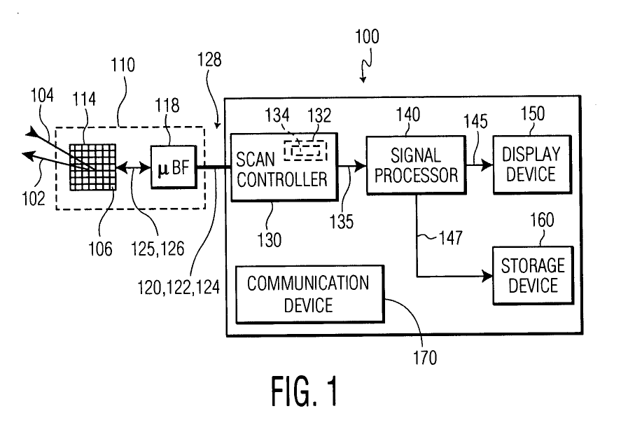

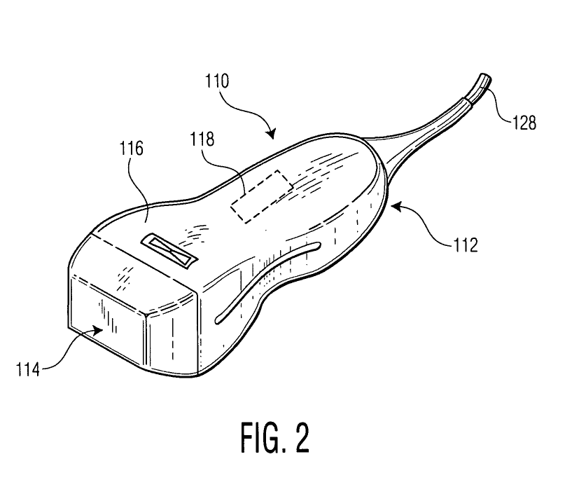

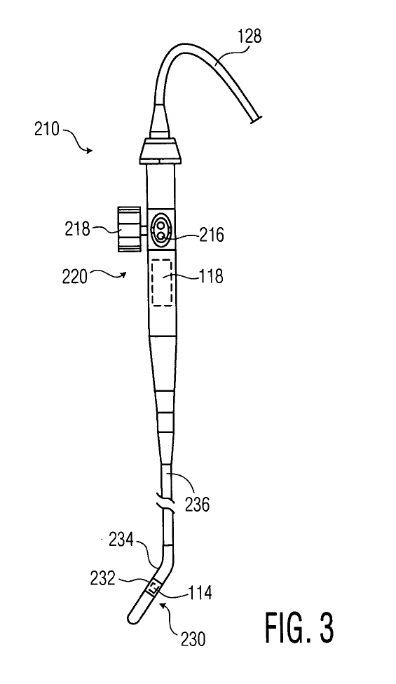

An ultrasonic imaging system according to the present invention is illustrated in FIG. 1, and further described with specificity hereinafter. The ultrasonic imaging system 100 includes an ultrasonic probe 110 having a housing 112 (FIG. 2), an ultrasonic transducer assembly 114, a selector switch 116 (FIG. 2), and a microbeamformer 118 (shown in phantom in FIGS. 2-3).

The ultrasonic transducer assembly 114 includes a plurality of acoustic elements 106 arranged in a number of columns and rows for generating at least one acoustic transmit beam 102 and/or receiving echoes from at least one receive beam 104. While the beams 102 and 104 are shown in the figure to be separated in space, it is understood by those skilled in the art that for a given scan line, the transmit and receive beams generated therein are substantially congruent. Advantageously, the ultrasonic transducer assembly 114 is capable of producing one or more acoustic transmit beams 102 in different directions and/or receiving echo signals from one or more receive beams 104 from different directions, thereby allowing the ultrasonic imaging system 100 to acquire ultrasound images while minimizing movement of the ultrasonic probe 110. A plurality of scan lines, each containing one transmit beam and at least one receive beam, produce ultrasonic data that together are processed into a displayed image. The plurality of scan lines is typically arranged in a planar format, such as a sector with apex at the center or behind the center of transducer assembly 114, with scan lines placed at regular angular displacements across the sector. The plurality of scan lines may alternatively be arranged in other formats, including cones, trapezoids, frustums, etc., to achieve interrogation of volumes in space, again with scan lines typically located at regular or irregular angular and/or spatial displacements. The acoustic elements 106 are preferably configured and arranged in a generally planar configuration, although other configurations and arrangements, such as convex or concave three-dimensional arrays are contemplated. Three-dimensional arrays give the advantage of expanding the practical field of view of the array, while still allowing arbitrary placement of scan lines within the field of view. Each acoustic element 106 is typically manufactured from a suitable piezoelectric material and is capable of generating an acoustic pulse at a particular frequency from a range of operable frequencies when a driver signal is applied to the acoustic element 106. In the transmit phase of a scan line, a number of acoustic pulses emanating nearly simultaneously from a plurality of acoustic elements 106 combine to form the acoustic transmit beam 102 for impinging upon an acoustic target. The ultrasonic imaging system 100 has a scan controller 130 for generating a composite drive/control signal 122 connected to microbeamformer 118, for electronically steering and focusing the acoustic transmit beam 102 and receive beam 104. Preferably, the composite drive signal 122 includes a plurality of driver signals for actuating a predetermined number of acoustic elements 106 and also includes beamforming delays for transmit and receive microbeamforming. The relative delays of the transmitted acoustic pulses from each element are varied from element to element by the scan controller 130 so as to determine the focus and steering of the resulting acoustic beams 102 and 104.

At least some of the energy in the acoustic beam 102 is reflected back towards the transducer assembly 114 as an echo signal along receive beam 104. Each acoustic element 106 is capable of receiving the echo signal in receive beam 104 from the acoustic medium and propagating the echo signal to microbeamformer 118, which generates a corresponding microbeamformed output signal 120. Again, relative delays are applied by the scan controller 130 to the received echo signals in receive beam 104 from each acoustic element 106 before the received echo signals are summed into the composite receive signal 120. The receive delays are preferably adjusted continuously throughout the propagation of the acoustic pulse of transmit beam 102 and the corresponding reflections along receive beam 104, such that the reflections maintain continuous focus on the elements 106 of transducer assembly 114. Scan controller 130 is operatively coupled to microbeamformer 118 such that microbeamformer output signal 120, comprising a plurality of sub-array beamformed signal sums, is additionally beamformed within scan controller 130 to form fully beamformed signal 135. It is contemplated that a number of the acoustic elements 106 in the transducer assembly 114 may be “inactive” elements (i.e. not configured for generating acoustic pulses or receiving echo signals) while the remaining acoustic elements 106 are “active” elements (i.e. configured for generating an acoustic pulse and receiving an echo signal 104). Further, the set if “active” elements may be configured for transmit and receive phases of the scan line, such that one set is employed for transmit, and another for receive. This allows the beam profile of the transmit beam 102 and receive beam 104 to be controlled independently for each scan line. In addition, the ultrasonic imaging system 100 further includes a signal processor 140, a display device 150, a storage device 160, and a communication device 170 for communicating images, data, or control information to or from an external system.

Still referring to FIG. 1, the scan controller 130 is coupled to the ultrasonic probe 110 (shown in dashed lines) by a connecting means 128 for communicating the composite drive signal 122 and a control signal 124 to microbeamformer 118. Additionally, the connecting means 128 communicates a composite receive signal 120 from microbeamformer 118 to the scan controller 130. More specifically, the scan controller 130 is operatively coupled to the ultrasonic transducer assembly 114 through microbeamformer 118, for varying characteristics and properties of the generated acoustic transmit beam 102 and receive beam 104 as discussed in further detail hereinafter.

The scan controller 130 generates a plurality of driver signals that correspond to the number of acoustic elements 106 to be activated. These driver signals are combined to form the composite drive signal 122 and are communicated to the transducer assembly 114. The scan controller 130 further controls the timing of the respective driver signals that are applied to the acoustic elements 106 (i.e. phase shifting) by means of control signal 124 connected to the microbeamformer 118. The scan controller 130 further controls the timing of the receive signal from receive beam 104, also by means of control signal 124. Control signal 124 thus controls the beamforming performed by microbeamformer 118.

In a preferred embodiment, the scan controller 130 includes a user interface 132 and associated circuitry for controlling the timing of the transmit drive and receive signals in order to control the steering and focus of acoustic beams. It is further contemplated that a predetermined number of acoustic elements 106 in the ultrasonic transducer assembly 114 may be activated by the scan controller 130 simultaneously thereby forming an active aperture for each acoustic transmit beam 102 and a another active aperture for each receive beam 104. Advantageously, the user interface 132 is operable by an operator to adjust and/or control the beam steering and active apertures for acquiring the desired image. In addition, the user interface 132 is configured and adapted for affecting other aspects of the ultrasonic imaging system 100, such as starting and stopping the system, directing the image information to the display device 150, selecting imaging modes, receive gain, transmit power, Doppler velocity scale, directing the image information to the storage device 160, retrieving the image information from the storage device 160, etc.

More specifically, when the acoustic transmit beam 102 is initially formed, a number of the active acoustic elements 106 disposed in the ultrasonic transducer assembly 114 are actuated simultaneously by the corresponding number of drive signals contained in composite drive signal 122 from the scan controller 130. The drive signal 122 is input to microbeamformer 118, which forms microbeamformed composite transmit drive signal 125. The microbeamformed transmit drive signal 125 connects to elements 106 of transducer assembly 114, and actuates at least one of said elements in the transmit phase of a scan line. Similarly, composite receive signal 126 from elements 106 of transducer assembly 114 are fed back to microbeamformer 118 during the receive phase of a scan line, where they are microbeamformed to form composite receive signal 120. The set of active receive elements are similarly activated in the receive aperture according to signals from control signal 124. In one embodiment, the acoustic elements 106 of transducer assembly 114 are arranged in a number of rows and columns to form an array where the scan controller 130 activates a predetermined number of acoustic elements 106 in the rows and columns to form the acoustic transmit beam 102 and acoustic receive beam 104. Advantageously, the scan controller 130 phase shifts the drive signals of composite drive signal 122, phase shifts the receive signals of composite receive signal 120, and modifies the control signal 124 to the microbeamformer 118 in order to actuate the desired acoustic elements 106 and to focus and steer the transmit beam 102 and receive beam 104 of each scan line such that the desired set of scan lines is gathered in a scan frame to form an image. In addition to steering and focus of scan lines, scan controller 130 and the associated circuitry also apply the individual driver signals in the composite drive signal 122 to one or more of the following: the pulse frequency, number of cycles, transmit apodization, receive apodization, etc.

In one embodiment, the associated circuitry in the scan controller 130 generates the control signal 124 and composite drive signal 122 in response to selections made by the operator in the user interface 132. The user interface 132 includes one or more user operable controls such as a rocker switch, a button, a trackball, a touchpad, a thumbwheel, a pointing stick, etc. These user operable controls permit the user to control various features and aspects of the ultrasonic imaging system 100, such as field of view of the ultrasonic probe 110, selection of imaging modes, receive gain, transmit power, Doppler scale, etc. In turn, the control signal 124, in cooperation with the associated circuitry, and the composite drive signal 122 control microbeamformer 118 for both generating the microbeamformed transmit drive signal 125 to elements 106 and for processing acoustic composite receive signal 126 from elements 106, and for microbeamforming composite receive signal 126 into composite receive signal 120. In addition, the control signal 124 cooperates with the associated circuitry to control the timing of the driver signals and the active aperture, and thus controls the electronic steering of the acoustic transmit beam 102 and acoustive receive beam 104, and thus determines the field of the acquired image.

In configurations using multidimensional transducer assemblies or large transducer assemblies, the connection between the ultrasonic probe 110 and the scan controller 130 may include a large number of connecting cables (i.e. one cable for each acoustic element 106 to be activated). Therefore, it is advantageous to include the microbeamformer 118 inside the ultrasonic probe 110 to reduce the number of connections included in connecting means 128. An example of a microbeamformer is disclosed in commonly owned U.S. Pat. No. 6,102,863 to Pflugrath et al., the contents of which is hereby incorporated by reference in its entirety.

In a preferred embodiment, the control signal 124 and composite drive signal 122 are generated by scan controller 130 and associated circuitry within the imaging system 100 in cooperation with the selector switch 116. In turn, the control signal 124 and drive signal 122 communicate information from the scan controller 130 for generating the microbeamformed composite transmit signal 125 and for microbeamforming composite receive signal 126. The control signal 124 and the composite drive signal 122 include signal information that is accepted by the microbeamformer 118 for generating the requisite driver signals to be applied to the selected active acoustic elements 106 to generate the acoustic transmit beam 102 and to process the acoustic receive beam 104. Preferably, the microbeamformer 118 controls the time delays of the individual driver signals for controlling the characteristics of the resultant acoustic beams 102 and 104. More particularly, the control signal 124 includes digital coefficients for configuring the microbeamformer 118 for a particular scan line. The microbeamformer 118 uses the digital coefficients in the control signal 124 to control steering and focus of the acoustic beam 102, as well as one or more of the following: the pulse frequency, number of cycles, transmit aperture, transmit apodization, etc. The microbeamformer 118 also uses the digital coefficients in the control signal 124 to control steering and focus of the acoustic beam 104, as well as one or more of the following: receive apodization, parallel receive beam formation, etc. The composite drive signal 122 may include one or more analog components for controlling other aspects of the acoustic beam 102, such as gain, waveform shape, number of cycles per pulse, transmit apodization, and frequency. By controlling the characteristics of the composite drivesignal 125 applied to the active acoustic elements 106 in the ultrasonic transducer assembly 114, and the processing of the received signal 126, the microbeamformer 118, in cooperation with the selector switch 116 and the scan controller 130, adjust the composition and placement acoustic beams 102 and 104, and thus control the composition of the image formed therefrom.

Alternatively, a further embodiment is contemplated in which a portion of the beamforming is done in the probe by the microbeamformer 118 as previously discussed and the balance is done in the scan controller 130. In this alternate embodiment, the composite drive signal 122 includes analog components and the control signal 124 contains digital coefficients as discussed in detail hereinabove. Composite receive signal 120 contains a multiplicity of microbeamformed receive signals from predetermined sub-arrays of transducer assembly 114. Scan controller 130 takes composite receive signal as input and completes the beamforming of sub-array receive signals according to controls in user interface 132 in combination with selector switch 116.

After the acoustic beam 102 is generated by one of the above-mentioned embodiments, it impinges an acoustic target and generates the echo signal receive beam 104. The resultant echo signal 104 is received by the ultrasonic transducer assembly 114 and ultimately by the active acoustic elements 106 contained therein. A complete cycle includes a transmit phase wherein the outgoing acoustic beam 102 is generated and a receive phase wherein the resultant echo signal receive beam 104 is received from the acoustic target.

As illustrated in FIG. 2, the ultrasonic probe ideally includes the selector switch 116 that is user operable for controlling characteristics of the acquired image by controlling the generation and timing of the composite drive signal 122 and control signal 124. The selector switch thus provides local control of ultrasonic probe 110 as will described hereinafter. The selector switch 116 may be a rocker switch, a button, a trackball, a touchpad, a thumbwheel, a pointing stick, etc.

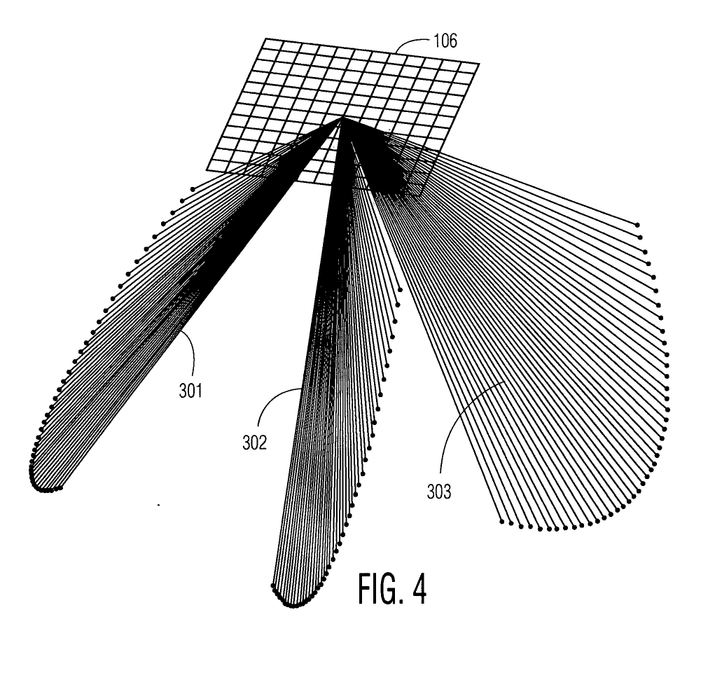

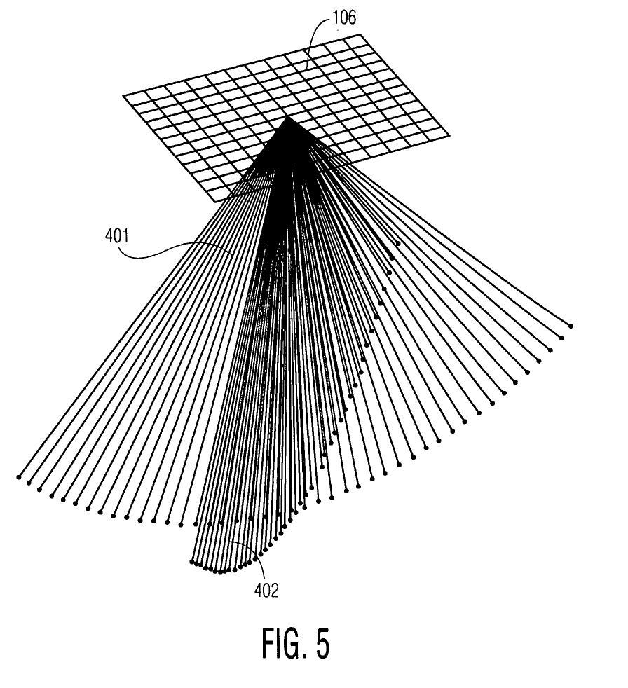

More particularly, when the user employs local control of the ultrasonic probe 110, that is, control of the probe by means of user interface 132 which is local to imaging system 100, the associated circuitry in the scan controller 130 generates the control signal 124 according to user selections on the user interface 132. Preferably, the user interface 132 includes at least one control device 134 having at least two positions or states for controlling the associated circuitry in response to the user's selections, including the selections of what functions are performed by selector switch 116. Optionally, the user interface 132 may include a number of control devices 134 for controlling the associated circuitry and/or other aspects of the scan controller's 130 operation in response to the user's selections. Each control device 134 may be a rocker switch, a button, a trackball, a touchpad, a thumbwheel, a pointing stick, etc. The control signal 124 has unique characteristics for each position or state of the control device 134. Therefore, by selecting a position on the control device 134, the user controls the associated circuitry for controlling the control signal 124 and the acquired image. For example, the operator can steer the planes of the scan in preselected modes such as lateral tilt, elevational tilt, or rotation. Referring to FIG. 4, three exemplar scan planes 301, 302, and 303 are shown in differing orientations of elevation tilt with respect to an exemplar 2-dimensional array of ultrasound probe elements 106. Each scan plane consists of a multiplicity of scan lines shown together forming a planar sweep. One of the scan planes, such as the central plane 302, may be scanned exclusively and repeatedly to form an image which is rendered in a 2-dimensional image on display device 150 of imaging system 100, or alternatively, more than one scan plane may be scanned alternately to form a composite image which is rendered in a 3-dimensional image on display device 150 of imaging system 100. The number of scan planes scanned and the relative positions of one or more scan planes are controlled by control signal 124 in response to user input on control device 134 of user interface 132. Referring now to FIG. 5, a similar arrangement of a multiplicity of scan planes 401 and 402 is in this case varied by degree of rotation with respect to each other. The number of scan planes scanned and the degree of rotation of one or more scan planes are controlled by control signal 124 in response to user input on control device 134 of user interface 132. It is understood by those skilled in the art that the arrangement of scan lines and planes exemplified in FIG. 4 and FIG. 5 is not limited, but may vary widely in line spacing, origin of scan lines, number of planes, orientation of planes, coplanarity of scan lines, etc. Further, control device 134 of user interface 132 may vary other imaging parameters of the scan lines as described hereinabove, such as gain, power, focus, apodization, etc.

By advantageously providing the selector switch 116 on the ultrasonic probe 110, and the microbeamformer 118 in the ultrasonic probe 110, the operator can readily control some of the operations of the ultrasonic imaging system 100 from the ultrasonic probe 110 and without accessing user interface 132 located on the system unit. When controlling the ultrasonic probe 110 remotely, the selector switch 116 in cooperation with the associated circuitry in the system scan controller 130 of imaging system 100 generates the control signal 124. Similar to local control of the ultrasonic probe 110 through user interface 132, when selector switch 116 is used, the associated circuitry in scan controller 130 generates the control signal 124 having unique characteristics for each position or state of the selector switch 116. Therefore, by selecting a position on the selector switch 116, the user controls the associated circuitry for controlling the control signal 124 and the acquired image. For example, the operator can steer the planes of the scan in preselected modes such as lateral tilt, elevational tilt, or rotation as shown in FIG. 4 and FIG. 5.

In the embodiment where the beamforming is done in the ultrasonic probe 110 (i.e. the ultrasonic probe 110 includes the microbeamformer 118), the selector switch 116 interacts with the associated circuitry of system scan controller 130 to generate the control signal 124 and the composite drive signal 122. In turn, the control signal 124 and composite drive signal 122 are communicated to microbeamformer 118. The resultant control signal 124 has the desired characteristics of beamforming digital coefficients and parameters for the selected position of selector switch 116. Likewise, the resultant composite drive signal 122 has the desired characteristics of delay and gain for the selected position on the selector switch 116. Therefore, by selecting a position on the selector switch 116, the user controls the microbeamformer 118 and the resultant acoustic beams 102 and 104. By selecting a particular position of the selector switch 116, the microbeamformer 118 generates the individual drive signals that are communicated to the ultrasonic transducer assembly 114. Additionally, the operation of the selector switch 116 may control one or more of the following characteristics of the composite drive signal 122: the pulse frequency, number of cycles, apodization, etc.

For example, the operator positions the ultrasonic probe 110 in contact between a patient's ribs, then holds the ultrasonic probe 110 stationary while electronically steering the scan using the same hand to operate the selector switch 116. In one embodiment, the control device 134 of user interface 132 of imaging system 100 and the associated circuitry may be actuated by the user to adjust the binding of selector switch 116 based on the mode of operation of the ultrasonic imaging system 100. Binding, as it is used in the present application, refers to a position or state of the selector switch 116 corresponding to a particular operation of the scan controller 130. For example, when using Flow mode or Doppler mode, the generated composite drive signal 122 and control signal 124, in response to actuation of selector switch 116, move the region of interest for one selected binding of selector switch 116, or vary the transmit power for another selected binding, or vary the tilt of the scan plane in yet a third selected binding. In a different imaging mode, such as Live 3D mode, the binding is selected such that selector switch 116 rotates the displayed volume, either by means of changing the composition of composite drive signal 122 and control signal 124 to control the scan line positions, or by changing display parameters communicated to signal processor 140 and a display device 150 in imaging system 100. The binding of the selector switch 116 may be predefined according to imaging mode, or alternatively may be user selectable wherein the clinician selects, for each imaging mode, the function associated with the various positions of the selector switch 116.

The connecting means 128 is generally a cable including a plurality of conducting elements, such as wires. Alternatively, the connecting means 128 can significantly be improved if some of the electronics are located in the ultrasonic probe housing 112 and the connecting means 128 is a wireless connection, such as infrared or radio frequency.

In another preferred embodiment, the ultrasonic imaging system 100 of FIG. 1 is operatively coupled to a TEE probe 210 that is illustrated in FIG. 3. TEE probe 210 includes a mid-handle 220, a distal portion 230, a selector switch 216, a positioner 218, and a connecting means 128. An example of a TEE probe is disclosed in commonly owned U.S. Pat. No. 6,572,547, the contents of which are hereby incorporated by reference in its entirety. The distal portion 230 includes an elongated section 236 attached to the distal end of the mid-handle 220, a flexible portion 234, and a distal region 232 that further includes the ultrasonic transducer assembly 114. Ideally, the TEE probe 210 includes a microbeamformer 118, as discussed previously, that is disposed within the mid-handle 220. The selector switch 216 is disposed on the mid-handle 220 along with the positioner 218.

The TEE probe 210 allows the clinician to readily access internal regions of the body for ultrasonic imaging. The flexible portion 234 is responsive to actuation of the positioner 218 by mechanical structures as is known in the art. By placing the distal portion 230 into a body cavity (i.e. the throat), the clinician positions the flexible portion 234 to a desired location for acquiring the ultrasonic image. The distal region 232 moves in conjunction with the flexible portion 234 thereby positioning the ultrasonic transducer assembly 114 accordingly. In configuration where a multidimensional transducer assembly 114 is included, the clinician advantageously combines the mechanical flexibility of the TEE probe 210 along with the electronic flexibility of the multidimensional transducer assembly 114 and the microbeamformer 118.

In further detail, the TEE probe 210 includes associated circuitry for cooperation with the selector switch 216. As discussed in detail in the previous embodiment, the selector switch 216 cooperates with the associated circuitry and scan controller 130 to generate the control signal 124 and composite drive signal 122. The control signal 124 and composite drive signal 122 are operatively coupled to the microbeamformer 118 which generates the individual drive signals applied to the acoustic elements 106 for generating the acoustic beams transmit 102 and receive 104. As in the previous embodiments using ultrasonic probe 110 of FIG. 2, beamforming may be performed within the TEE probe 210, within the ultrasonic imaging system 100, or as a combination. Receiving and processing of the echo receive signal 104 is similar to that discussed for ultrasonic probe 110. Advantageously, TEE probe 210 may be substituted for ultrasonic probe 110 in any of the previously discussed embodiments.

This composite receive signal 120 is communicated through the scan controller 130 (after completion of beamforming, if applicable, as described hereinabove) to the signal processor 140. In the signal processor 140, the composite receive signal 120 of the transducer assembly 114 is transformed by associated circuitry in the signal processor 140 to generate an image signal 145. A display device 150 is operatively coupled to an output of the signal processor 140 for receiving one or more image signals 145 and for transforming the image signals 145 into a video image. Essentially, the display device 150 is capable of displaying data corresponding to the at least one image signal 145. It is preferred that the display device 150 be a video or LCD monitor that is readily viewable by attending personnel.

Alternatively, the associated circuitry in the signal processor 140 produces a data signal 147 in addition to, or in lieu of the image signal 145. In an embodiment where signal processor produces the data signal 147 in addition to the image signal 145, it is preferred that the data signal 147 includes substantially identical information as contained in the image signal 145. A storage device 160 is operatively coupled to an output of the signal processor 140 for receiving one or more data signals 147 and for transforming the at least one data signal 147 into an organized sequence representing the information included in the at least one data signal 147. Essentially, the storage device 160 is capable of storing data corresponding to the at least one data signal 147. It is preferred that the storage device is a magnetic storage device such as a magnetic disc or a magnetic tape. More preferably, the storage device is a hard drive. It is contemplated that other storage devices such as optical storage devices and solid state nonvolatile memory devices such as FLASH memory may be used in lieu of the hard drive without departing from the scope or spirit of the present invention.

In another embodiment, the user interface 132 is further adapted and configured to cooperate with the associated circuitry in the signal processor 140 for retrieving the data stored in the storage device 160. In this embodiment, the storage device 160 transforms the stored data into at least one data signal 147 that is communicated to the associated circuitry of the signal processor 140. The associated circuitry of the signal processor 140 transforms the at least one data signal 147 into at least one image signal 145. The at least one image signal 145 is then communicated to the display device 150 for viewing as previously discussed.

The described embodiments of the present invention are intended to be illustrative rather than restrictive, and are not intended to represent every embodiment of the present invention. Various modifications and variations can be made without departing from the spirit or scope of the invention as set forth in the following claims both literally and in equivalents recognized in law.

1. An ultrasonic imaging apparatus comprising:
a scan controller;
ultrasound processing electronics; and
an ultrasonic transducer probe electrically connected to the scan controller and the ultrasound processing electronics and having a selector switch and a housing, the transducer probe including: a multidimensional transducer assembly capable of generating at least one acoustic beam and/or receiving at least one echo signal and a microbeamformer for controlling aspects of said generating and receiving at the transducer probe, wherein associated control circuitry disposed within the transducer probe and operatively coupled to said ultrasonic transducer array is included for providing direct user control of the at least one acoustic beam, and wherein said selector switch is constructed to communicate with at least one of at least two user-selectable states to the scan controller for controlling the associated circuitry in accordance with the at least one communicated state, wherein the at least two user-selectable states are defined by the user.

2. The ultrasonic imaging apparatus of claim 1, wherein the at least one user-selectable state adjusts a characteristic of the at least one acoustic beam.

3. The ultrasonic imaging apparatus of claim 1, wherein the selector switch is selected from the group consisting of a rocker switch, a button, a trackball, a touchpad, a thumbwheel, and a pointing stick.

4. The ultrasonic imaging apparatus of claim 1, wherein the associated circuitry generates a control signal according to the at least one communicated state, and said control signal controls at least one characteristic of the at least one acoustic beam.

5. An ultrasonic imaging system comprising:
a scan controller;
ultrasound processing electronics; and
an ultrasonic imaging probe connected to the scan controller and the ultrasound processing electronics, the ultrasonic imaging probe including:
a housing,
a multidimensional transducer assembly configured and adapted to fit within said housing, the multidimensional transducer assembly capable of generating at least one acoustic beam and/or receiving at least one echo signal,
a microbeamformer for controlling aspects of said generating and receiving at the multidimensional transducer assembly, wherein associated circuitry is disposed within the ultrasonic probe and is operatively coupled to the ultrasonic transducer assembly for providing direct control of the at least one acoustic beam, and
a selector communicating at least one of at least two user-selectable states to the scan controller for controlling the associated circuitry in accordance with the at least one communicated state, wherein the at least two user-selectable states are defined by the user; and
means within said scan controller for producing a drive signal for operating the microbeamformer for generating the at least one acoustic beam in accordance with the at least one communicated state.

6. The ultrasonic imaging system of claim 5, further comprising:
a signal processor coupled to said ultrasonic transducer assembly for processing the at least one echo signal, thereby forming at least one image signal;
means for connecting said ultrasonic probe to an ultrasonic imaging apparatus; and
a display for displaying the at least one image signal.

7. The ultrasonic imaging system of claim 5, wherein the at least one user-selectable state adjusts a characteristic of the at least one acoustic beam.

8. The ultrasonic imaging system of claim 5, wherein the selector is selected from the group consisting of a rocker switch, a button, a trackball, a touchpad, a thumbwheel, and a pointing stick.

9. The ultrasonic imaging system of claim 5, wherein the scan controller further includes another selector having at least two positions and operatively coupled to the associated circuitry of the scan controller for adjusting at least one characteristic of the at least one acoustic beam.

10. The ultrasonic imaging system of claim 5, wherein the associated circuitry generates a control signal according to the at least one communicated state, and said control signal controls at least one characteristic of the at least one acoustic beam.

11. A signal generated by an ultrasonic imaging system and relaying control information in accordance with a state selected by a selector of an ultrasonic imaging probe for controlling the output of at least one acoustic beam generated by said ultrasonic imaging system, said system comprising:
a scan controller;
a multidimensional transducer assembly configured and adapted to fit within said probe, the multidimensional transducer assembly capable of generating the at least one acoustic beam and/or receiving at least one echo signal;
a microbeamformer for controlling aspects of said generating and receiving at the multidimensional transducer assembly, wherein associated circuitry is disposed within the ultrasonic probe and is operatively coupled to the ultrasonic transducer assembly for direct control of the at least one acoustic beam; and
a selector communicating at least one of at least two user-selectable states to the scan controller for controlling the associated circuitry in accordance with at least one of the communicated states, wherein the at least two user-selectable states are defined by the user; and
means within said scan controller for producing a drive signal for operating the microbeamformer to generate the at least one acoustic beam.

12. An ultrasonic probe comprising:
a housing;
a multidimensional transducer assembly configured and adapted to fit within said housing, the multidimensional transducer assembly capable of generating at least one acoustic beam and/or receiving at least one echo signal;
a microbeamformer for controlling aspects of said generating and receiving at the multidimensional transducer assembly, wherein associated circuitry is disposed within the ultrasonic probe and is operatively coupled to the ultrasonic transducer assembly for direct control of the at least one acoustic beam; and
a selector communicating at least one of at least two user-selectable states to the associated circuitry for controlling the associated circuitry in accordance with at least one of the communicated states, wherein the at least two user-selectable states are defined by the user.

13. The ultrasonic probe of claim 12, wherein a scan controller is operatively coupled to said associated circuitry, said scan controller producing a drive signal for operating the ultrasonic transducer assembly to generate the at least one acoustic beam.

14. The ultrasonic probe of claim 12, wherein the at least one user-selectable state adjusts a characteristic of the at least one acoustic beam.

15. The ultrasonic probe of claim 12, wherein the selector switch is selected from the group consisting of a rocker switch, a button, a trackball, a touchpad, a thumbwheel, and a pointing stick.

16. The ultrasonic probe of claim 12, wherein the associated circuitry generates a control signal according to the at least one communicated state, said control signal controls at least one characteristic of the at least one acoustic beam.
